(19) United States
(12) Patent Application Publication
(10) Pub. No.: US 2024/0335116 A1
(43) Pub. Date: Oct. 10, 2024

(54) METHODS FOR NOISE REMOVAL IN FUNCTIONAL MRI USING SPECTRALLY SEGMENTED REGRESSION OF MOTION PARAMETERS AND PHYSIOLOGICAL NOISE

(71) Applicant: UNM RAINFOREST INNOVATIONS, Albuquerque, NM (US)

(72) Inventors: Stefan POSSE, Albuquerque, NM (US); Khaled TALAAT, Albuquerque, NM (US)

(73) Assignee: UNM RAINFOREST INNOVATIONS, Albuquerque, NM (US)

(21) Appl. No.: 18/579,540

(22) PCT Filed: Jul. 28, 2022
(86) PCT No.: PCT/US2022/038740

§ 371 Date: Jan. 16, 2024

Related U.S. Application Data
(60) Provisional application No. 63/229,379, filed on Aug. 4, 2021.

(51) Int. Cl. A61B 5/00; A61B 5/055; G01R 33/48; G01R 33/56; G01R 33/565

(52) U.S. Cl. CPC A61B 5/0042; A61B 5/055; A61B 5/7207; A61B 5/7257; G01R 33/4806; G01R 33/5608; G01R 33/56509; A61B 2576/026

(57) ABSTRACT

A method of performing functional magnetic resonance imaging that provides linear nuisance regression relying on spectral and temporal segmentation of the motion parameters, the physiological noise signals, and hardware related signal fluctuations; and provides a technique that both reduces data losses and improves the suppression of physiological noise and motion in resting-state fMRI. The technique minimizes the loss of intrinsic resting-state signal fluctuations and mitigates the introduction of false positive signals through segmentation of the regression vectors in the spectral domain.

### RELATED APPLICATIONS

This application is a U.S. National Phase application of PCT/US2022/038740 filed Jul. 28, 2022, which claims priority to U.S. Provisional Application No. 63/229,379, filed on Aug. 4, 2021, the disclosures of which are incorporated by reference herein in their entireties.

### STATEMENT REGARDING FEDERALLY SPONSORED RESEARCH & DEVELOPMENT

This invention was made with government support by the NIH grant No. R21 EB022803. The government has certain rights in the invention.

### BACKGROUND OF THE INVENTION

Studies have demonstrated task-related fMRI signal changes and spatially coherent signal fluctuations in resting-state fMRI at frequencies above 0.3 Hz. However, it has been shown that whole-band linear nuisance regression used in high-frequency resting-state fMRI can result in the introduction of artifactual connectivity in the high-frequency regime.

Resting-state functional MRI (rsfMRI) has emerged as an adjunct to task-based fMRI, allowing for simultaneous mapping of functional connectivity across dozens of resting-state networks (RSNs) to study a wide range of neuroscience and clinical applications with more than one hundred resting-state networks identified in group studies and interindividual differences in network topology detectable in individual subjects. Advances in high-speed fMRI, which enable unaliased sampling of physiological signal fluctuations, have increased sensitivity for mapping functional connectivity and detecting dynamic changes compared with conventional echo-planar imaging (EPI) techniques. Recently, several studies using high-speed fMRI techniques have reported different potential resting-state networks (RSNs) at high frequencies (up to 5 Hz) suggesting that functional integration between brain regions at rest occurs over broader frequency bands than previously thought.

The majority of published studies on high-frequency resting-state fMRI (hfrsfMRI) has utilized nuisance regression. Concerns have been raised on the use of conventional regression techniques in the preprocessing of rapidly sampled fMRI data. In particular, it was demonstrated that the use of whole-band linear regression in motion and physiological noise correction can pass structured network patterns from the conventional low frequency band artificially to higher frequencies and introduces artifactual high frequency connectivity above those predicted from the canonical model which sets an upper limit at 0.3 Hz. Alternate methods such as conventional FIR filtering suffer from extensive data losses as large portions of the spectra may be contaminated with respiration, cardiac pulsation, and their harmonics.

Physiological noise (primarily due to respiration and cardiac pulsation) typically encompasses bands of frequencies and regressing respiratory variation and heart rate variability can significantly alter functional connectivity maps of the default mode network. Regression over shorter time intervals may be exploited to reduce the loss of resting-state signal fluctuations or introduction of false positive signal during as the physiological noise bands are narrower in shorter intervals. It motivated the recent development of harmonic regression with autoregressive noise (HRAN) using temporally segmented regression, which has been shown to remove physiological noise while leaving the neural signal intact, thereby increasing detection of task-driven voxels. However, application of HRAN to high-speed resting-state fMRI have not been investigated.

Resting-state fMRI is highly sensitive to movement which obscure networks as well as create false positive correlations. Motion regression utilizing up to 24 realignment parameters, spike regression, motion scrubbing, PCA based regression of nuisance signals using aCompCor and a range of ICA based approaches have been developed to remove motion-related artifacts in resting-state fMRI. However, the applicability of these approaches to high-frequency resting-state fMRI remains insufficiently characterized.

### BRIEF SUMMARY OF THE INVENTION

In one embodiment, the present invention provides linear nuisance regression relying on spectral and temporal segmentation of the motion parameters, the physiological noise signals, and hardware related signal fluctuations.

In other embodiments, the present invention provides a technique that both reduces data losses and improves the suppression of physiological noise and motion in resting-state fMRI. The technique minimizes the loss of intrinsic resting-state signal fluctuations and mitigates the introduction of false positive signals through segmentation of the regression vectors in the spectral domain.

In other embodiments, the present invention provides a technique that exploits the fact that physiological noise tends to have narrower spectral bands over shorter time periods due to the decreased variability and relies on segmenting the scan data in time into n segments within which frequency and amplitude fluctuations of motion parameters and physiological noise are reduced compared to the entire scan.

In other embodiments, the above method is implemented using a sliding window in the time domain.

In other aspects, the present invention provides an approach for the suppression of motion and physiological noise in high-frequency resting-state fMRI. The approach improves the suppression of noise and avoids the introduction of spurious components due to shared regression weights between different parts of the spectral domain when sufficient number of spectral bands of the regression vectors is used.

In another embodiment, the present invention provides a method of performing functional magnetic resonance imaging, comprising the steps of: a. Measuring and reconstructing a time series of high-speed functional MRI scan data sets in human or animal brain using an acquisition rate that enables spectral separation of physiological noise signals, including, but not limited to respiratory and cardiac pulsatility, and hardware related signal fluctuations; b. Measuring a plurality of rigid body movement parameters for translations and rotations in different directions in the data sets relative to one of the data sets; c. Spatially segmenting the data sets into multiple brain regions, including, but not limited to gray matter, white matter and cerebrospinal fluid containing regions; d. Measuring the physiological noise signals and the hardware related signal fluctuations in the regions; e. Spectrally and temporally segmenting the motion parameters, the first derivatives of the plurality of rigid body movement parameters, the physiological noise signals and the hardware related signal fluctuations, resulting in multiple regression vectors for each temporal segment and each of the regions; f. Temporally segmenting the functional MRI scan data sets into the temporal segments; and g. Performing linear regression within each temporal segment and spatial region of the data sets using the regression vectors.

Steps may include the following: using a minimum acquisition rate that corresponds to Nyquist frequency for measuring cardiac pulsatility; minimizing the loss of intrinsic resting-state signal fluctuations and mitigating the introduction of false positive signal fluctuations by the segmentation of the regression vectors in the spectral and temporal domain; segmenting the scan data in time into n segments within which frequency and amplitude fluctuations of motion parameters and physiological noise are reduced compared to the entire scan; employing a temporally segmented regression of the scan data using spectral segmentation of the motion parameters, the physiological noise signals and the hardware related signal fluctuations into k spectral segments with no overlap; the scan data is segmented in time into n segments with no overlap and the length of each segment is chosen such that it is long enough to resolve features in the spectral domain and short enough for respiration rate, cardiac pulsation and hardware related signal fluctuations, to be sufficiently stable; and employing a sliding window for temporal segmentation.

Other methods to enhance the effectiveness of the present invention also include the following: using regression vectors constructed from the motion parameters and their higher order derivatives wherein each motion parameter is filtered into k number of segments in the spectral domain to obtain an independent regression vector for each spectral band; using a non-causal filter to filter the motion parameters, the physiological noise signals and the hardware related signal fluctuations by applying FFT to the motion parameter time course, the physiological noise signal time courses and the hardware related signal fluctuation time courses, zeroing the spectral components outside of the spectral band of interest, then using inverse FFT to obtain the segment of interest; using a temporally segmented regression of the filtered motion parameters to obtain motion-corrected data; generating using a spatial mask of the labeled feature based on an anatomical brain atlas and used to obtain a spatially averaged signal to construct a representative regression vector; generating a mask based on a power-spectral integral threshold relative to a labeled non-physiological noise frequency range; for the average signal within the mask, pass-band filtering in the frequency range of the labeled feature to construct a regression vector for the feature within the segment; phase shifting individually constructed regression vectors for each slice of the functional MRI scan data sets to minimize the power spectral integral in the frequency range of the feature in the corrected signal across the entire slice; determining the maximum number of spectral bands using self-regression testing where regression vectors are segmented into a varying number of spectral bands and regressed using each set of spectrally segmented regression vectors with the maximum number of spectral bands decided based on the tolerated residual correlation between the original regression vector and resulting regressed signal.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 shows a seed-based correlation analysis applied to simulated resting-state fMRI data that represent two 2-node resting-state networks in two different frequency ranges below (seed regions i and ii) and above 0.3 Hz (seed regions iii and iv). (a) Seed regions i to iv. (b,c) Original Rician brain model without noise added. (d,e) Brain model with noise from in-vivo scan motion parameters added, which introduces false positive correlations. LP-Noise: low-pass filtered noise, WB-noise: whole bandwidth noise. Correlation threshold: 0.1.

FIG. 2 shows the dependence of spectrally segmented regression of simulated resting-state fMRI data, with noise from in-vivo scan motion parameters added, on the number of spectral bands of the regression vectors in two different frequency ranges below and above 0.3 Hz. (a,b) Conventional regression using 1 spectral band (c,d) Spectrally segmented regression using 3 spectral bands of 0.81 Hz bandwidth. (e,f) Spectrally segmented regression using 9 spectral bands of 0.27 Hz bandwidth. False positive correlations decrease with increasing spectral segmentation. LP-Noise: low-pass filtered noise, WB-noise: whole bandwidth noise. Correlation threshold: 0.1.

FIG. 3 is a self-regression test to examine the effectiveness of spectrally segmented regression: (a) the simulated signal, which contains a range of low and high frequency features, (b) correlation of the simulated signal with the regressed signal as a function of the number of spectral segments of the simulated signal as regression vectors.

FIG. 4 is a comparison of physiological noise regression in HRAN and the spectrally and temporally segmented regression implemented in the TurboFilt software using the demonstration data provided with HRAN acquired with a TR of 0.347 sec. The top figure shows spectra from a visual cortex seed before (Original Data) and after physiological noise regression using HRAN and spectrally and temporally segmented regression implemented in TurboFilt. Spectrograms are shown for (a) original data with physiological noise, (b) HRAN corrected data, (c) data corrected using spectrally and temporally segmented regression.

FIG. 5 is a comparison of physiological noise regression using spectrally and temporally segmented regression and HRAN in 3 regions of interest for a healthy control subject during hyperventilation. Frequency spectra are shown for (a) a visual network ROI, (b) a default mode network ROI, (c) a CSF ROI, and (d) motion parameters. The embedded figure in (a) shows a zoomed in view of the denoised signal in the visual network after spectrally and temporally segmented regression. Data were acquired using MS-EVI with TR: 246 ms and a scan time of 4:35 min.

FIG. 6 illustrates an application of spectrally and temporally segmented regression to high-speed resting-state fMRI data acquired in a healthy control for full bandwidth (b-e) and high frequency correlations (f-i) using (a) seeds in the auditory network (AUN), the default mode network (DMN), and the visual network (VSN). (b-e) Comparison of whole-band correlations: (b) No motion regression, no high pass filtering, (c) Full-width, whole-band motion regression, no high pass filtering, (d) Full-width, whole-band motion regression with high pass with 0.005 Hz stop-band and 0.03 Hz pass-band limit, (e) spectrally and temporally segmented regression (8 time segments and 12 spectral segments) with no FIR high pass filtering. (f-i) Comparison of correlations above 0.3 Hz using seeds in the auditory network (AUN), the default mode network (DMN), and the visual network (VSN): (f) no motion regression, (g) full width motion regression, (h) spectrally segmented regression (12 segments), (i) spectrally and temporally segmented regression (12 spectral segments and 8 temporal bands). Data were acquired using MS-EVI with TR: 246 ms and a scan time of 4:35 min. Correlation thresholds: 0.3 for (b-e) and 0.25 for (f-i).

FIG. 7 is a comparison of physiological noise regression in resting-state networks of a healthy control using spectrally and temporally segmented regression and HRAN: (a) Seed ROIs. (b) Spectrally and temporally segmented regression (all frequency range), (c) HRAN (all frequency range) and high pass >0.03 Hz but without TurboFilt-based motion regression, (d) HRAN (all frequency range) after spectrally and temporally segmented motion regression (12 segments and 8 temporal bands) in TurboFilt, (e) Spectrally and temporally segmented regression (>0.3 Hz), (f) HRAN (>0.3 Hz). Data were acquired using MS-EVI with TR: 246 ms and a scan time of 4:35 min. A correlation threshold of 0.3 is applied to b-d, while a threshold of 0.25 is applied to e and f.

FIG. 8 is a comparison of physiological noise regression in resting-state networks of a healthy control using averaged sliding window correlation in addition to preprocessing with either spectrally and temporally segmented regression or HRAN. (a) Unilateral auditory network, default mode network and sensory motor network seed regions. (b) Low-frequency (<0.3 Hz) connectivity maps. The high frequency (>0.3 Hz) correlation analysis using preprocessing with (c) HRAN and (d) temporally and spectrally segmented regression of motion parameters and physiological noise. Low frequency maps threshold at 0.6. High-frequency AUN and DMN maps threshold at 0.3, while SMN is threshold at 0.2. Data were acquired using MS-EVI with a scan time of 4:35 min.

FIG. 9 is a comparison of artifactual connectivity in a healthy control during hyperventilation (compare with FIG. 5) using averaged sliding window correlation in addition to preprocessing with either spectrally and temporally segmented regression or HRAN. (a) Seed regions in CSF and WM. (b) Low-frequency connectivity maps threshold at correlation value of 0.4. High frequency connectivity maps (>0.3 Hz) obtained with (c) HRAN and (d) temporally and spectrally segmented regression of motion parameters and physiological noise. High frequency correlation maps threshold at 0.3. Data were acquired using MS-EVI with a scan time of 4:35 min.

FIG. 10 illustrates the detection of high frequency connectivity in a patient with a brain tumor using averaged sliding window correlation in addition to preprocessing with either spectrally and temporally segmented regression or HRAN. (a) Seed regions in Broca's area, auditory cortex, WM and CSF. (b) Low-frequency connectivity maps. Correlation thresholds for the language network, the auditory networks, WM and CSF are 0.4, 0.4, 0.35 and 0.35, respectively. High frequency (>0.3 Hz) connectivity analysis using (c) HRAN and (d) temporally and spectrally segmented regression of motion parameters and physiological noise. Correlation thresholds for the language network, the auditory networks, WM and CSF are 0.2, 0.2, 0.1 and 0.1 respectively. Data were acquired using MB-EPI with a scan time of 10:21 min.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure, or system. Further, the terms and phrases used herein are not intended to be limiting, but rather to provide an understandable description of the invention.

In one embodiment, the present invention provides a linear nuisance regression relying on spectral and temporal segmentation of the motion parameters and the physiological noise signals. The embodiment is shown in simulations and analysis of in vivo resting-state multi-slab echo-volumar-imaging and multi-band EPI data with TR as short as 205 ms to not only avoid the injection of artifactual connectivity, but it also substantially improves the removal of motion effects and physiological noise throughout the whole frequency spectrum even when uncertainties are present in part of the spectral domain of the regression vectors. Connectivity at high frequencies in 5 healthy controls and 2 patients with brain tumors was consistent with that in traditional low-frequency networks. A comparison with recently developed temporally segmented Harmonic-Regression-with-Autoregressive-Noise (HRAN) shows that the new approach is particularly powerful for regressing nuisance signals that cover a broad frequency spectrum. Combination of this regression approach with averaged sliding-window correlation further enhances confound suppression and improves mapping of resting-state networks at frequencies above 0.3 Hz.

### METHODS

### Spectrally and Temporally Segmented Regression

In a preferred embodiment, the present invention employs temporally segmented regression of the scan data using spectrally segmented nuisance parameters. The scan data is segmented in time into n segments with no overlap. The length of each segment is chosen such that it is long enough to resolve features in the spectral domain and short enough for respiration rate and cardiac pulsation to be sufficiently stable. In case the data is not divisible into n segments, the remainder images are discarded from the beginning or end of the scan.

The regression process involves the generation of a set of regression vectors, [symbol] , temporal segmentation of the data and regression vectors, calculation of regression coefficients, and subtraction of the regression series from the original signal, [symbol] ^{j}, to obtain the corrected signal, [symbol] ₛ^{j}, as expressed in Equation 1.

$${\vec{x}}_{s}^{j} = {\vec{x}}^{j} - {\sum }_{i = 1}^{{L}^{j}} {\vec{γ}}_{i}^{j} {\vec{S}}_{i}^{j} \qquad ( 1 )$$

where j is an index for the temporal segment, [symbol] ᵢ^{j} a calculated weighting coefficient, and L^{j} is the number of regression vectors within segment j. The corrected signal in a voxel is the concatenation of [symbol] ₛ^{j} in time as expressed in Equation 2.

$$\vec{{x}_{s}} = [ {\vec{x}}_{s}^{j = 1} , {\vec{x}}_{s}^{j = 2} , … , {\vec{x}}_{s}^{j = n} ] \qquad ( 2 )$$

The coefficients of regression can be obtained by minimizing the quadratic form | [symbol] ^{j}−S^{j} [symbol] ^{j}|² as explained by Gembris et al. Magnetic Resonance in Medicine 2000 for the conventional approach. This minimization results in Equation 3.

$$( {S}^{{j}^{T}} {S}^{j} ) {\vec{γ}}_{i}^{j} = {S}^{{j}^{T}} {\vec{x}}^{j} \qquad ( 3 )$$

### Regression of Motion Parameters

Regression vectors are constructed from motion parameters measured experimentally during the scan for translations and rotations in different directions. Each motion parameter is filtered into k number of segments in the spectral domain to obtain an independent regression vector for each spectral band. A non-causal filter is used to filter the motion parameters by applying FFT to the motion parameter time course then using inverse FFT to obtain the segment of interest. Temporally segmented regression of the filtered motion parameters is then applied to obtain motion-corrected data. Spectral segmentation of motion parameters allows for independent regression in different spectral bands which mitigates the injection of artifactual connectivity when uncertainties in the regression vectors are present in a frequency range. Spectral bands of uniform or non-uniform bandwidth can be used. In case the spectral domain is divided into bands of uniform width, the number of bands must be such that bands of interest in post-processing are independent of other bands that may introduce artifactual connectivity into the bands of interest. In addition, regression vectors can be constructed from higher order derivatives of these motion parameters.

Using a large number of narrow bands, relative to width of bands of interest, can make it easy to meet this independence criterion but may compromise the effectiveness of the regression process if an excessive number of bands is used as discussed later in self-regression tests in the simulations. In addition to nuisance regressors, the set of motion regression vectors always includes a constant vector of ones. This step is equivalent to subtraction of the average of each signal prior to regression and is necessary to zero-center the signals for regression.

Regression of Physiological Noise

In other embodiments, the present invention reduces data losses during regression of physiological noise by employing temporal segmentation of the signals. After the motion-corrected data is divided into temporal segments, respiratory and the cardiac noise in each segment is labeled in the frequency domain. No frequency segmentation within the labeled physiological noise bands is performed. A spatial mask of the labeled feature is generated and used to obtain a spatially averaged signal to construct a representative regression vector. The mask is generated based on a power-spectral integral threshold relative to a labeled non-physiological noise frequency range. The average signal within the mask is pass-band filtered in the frequency range of the labeled feature to construct a regression vector for the feature within the segment. The constructed regression vectors are phase shifted individually for each slice such to minimize the power spectral integral in the frequency range of the feature in the corrected signal across the entire slice. Iterative minimization is used to search for optimal phase shifts, constrained to an arbitrary range. This phase shifting approach accounts for inter-slice time delays in physiological signal pulsation. In addition to nuisance regressors, the set of physiological noise regression vectors always includes a constant vector of ones. This step is equivalent to subtraction of the average of each signal prior to regression and is necessary to zero-center the signals for regression.

In addition, hardware related signal fluctuations in different frequency bands can be regressed using the above approach. The above method can also be applied using a sliding window in the time domain for temporal segmentation.

The present method was implemented into an in-house developed MATLAB-based framework for the post-processing of fMRI scans named TurboFilt. Data in TurboFilt were processed without spatial smoothing.

### Simulations

Simulations were conducted to verify that the proposed method mitigated the injection of artifactual connectivity into higher frequencies and effectively recovered the original signals prior to injection of noise. A 2-slice Rician brain model was generated with an intensity range from 0 to 5. Each generated signal consisted of 3000 points with a TR of 0.205 sec resulting in a frequency bandwidth of 2.43 Hz. A first network consisted of two seed regions defined in anterior brain regions that were correlated at frequencies >0.3 Hz while a second network consisted of two seed regions defined in posterior brain regions that were correlated at frequencies <0.3 Hz. This was achieved by mixing the signals associated with the voxels under the seeds with a Rician correlation vector that was either high passed (>0.3 Hz) or low passed (<0.3 Hz) with a mixing coefficient, β, of 0.3 according to Equation 4.

$${s}_{1} = ( 1 - β ) {s}_{2} + β {s}_{2} \qquad ( 4 )$$

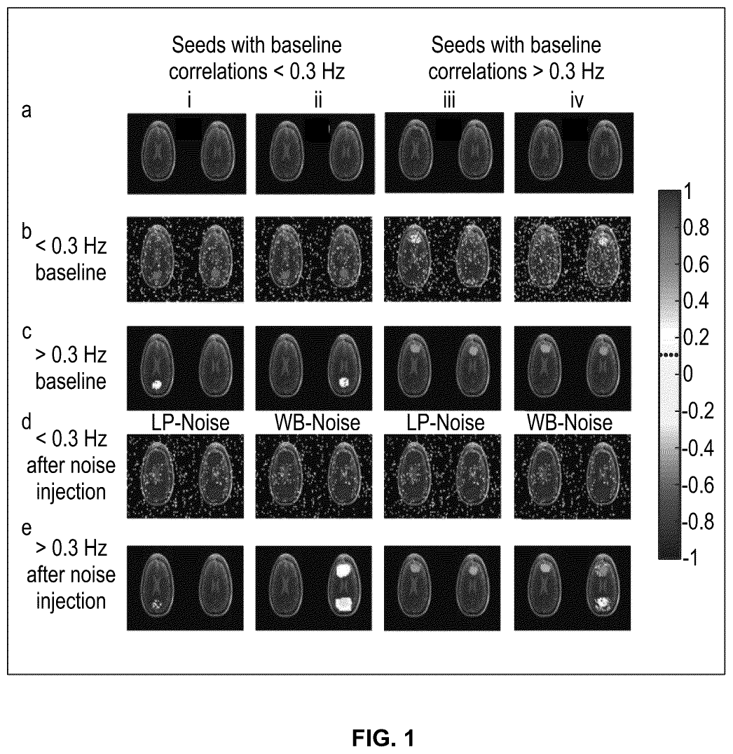

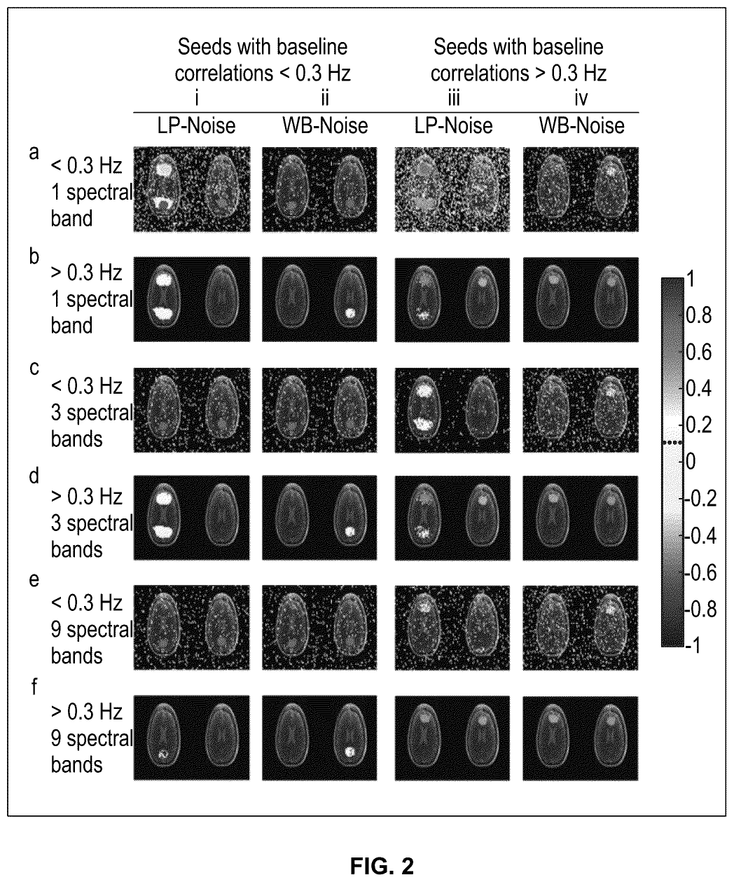

Motion parameters from an actual scan were introduced as noise in the simulations. Low-pass filtered (<0.3 Hz) translation parameters (tx, ty, and tz) were injected into two seed regions on the left slice as shown in FIGS. 1 and 2 using mixing coefficients of 0.3, 0.4, and 0.5, respectively, to create a mismatch with non-filtered translation parameters that were injected into regions containing the seeds in the right slice using the same mixing coefficients as in the left slice. Regression vectors were constructed from spectral segmentation of the original non-filtered translation parameters. The number of spectral segments was varied to understand the effect of spectral segmentation on regression. Denoised signals were compared to signals prior to the injection of noise to assess the effectiveness of the regression technique.

### Self-Regression Tests

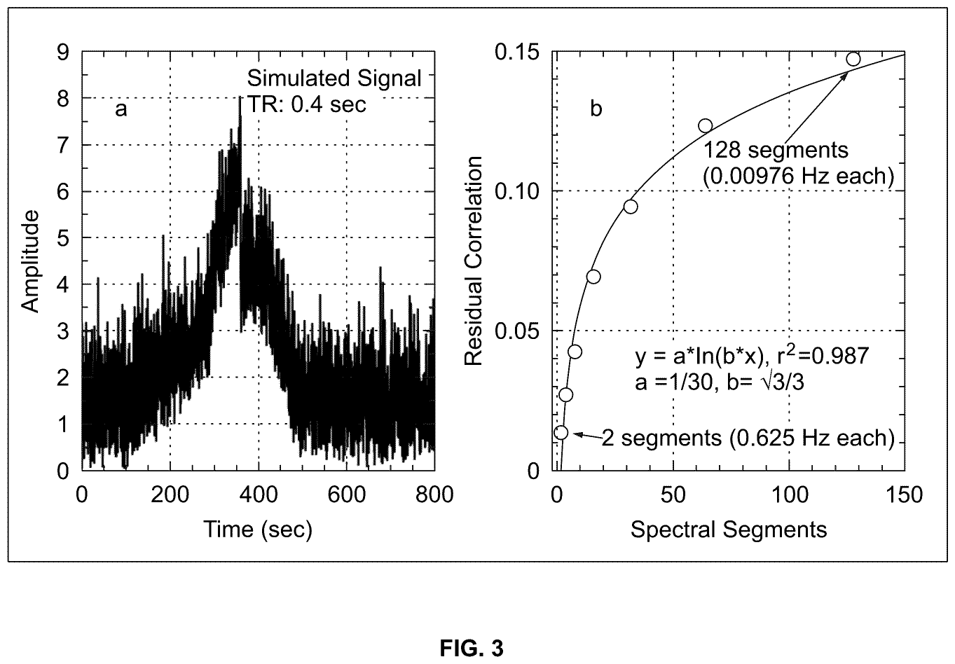

Additional simulations were conducted to further understand the effect of the bandwidth of the regression vectors on the effectiveness of the regression process. A Rician signal (TR: 0.4 sec, 2000 points) was generated and confounding features were added to the signal (FIG. 3a). The same signal was then used to construct the regression vectors. It is, therefore, expected that the regressed signal would be a constant zero with no correlations with the original signal in the event of successful regression. In the event of failure of the regression process, the Pearson correlation coefficient between the original signal and the regressed signal would be 1 indicating that regression had no effect on the signal. Therefore, residual correlations between the simulated signal and the regressed signal can provide an indicator of the effectiveness of the regression process when different numbers of regression vectors representing different spectral bands in the original signal are used. Residual correlations closer to zero indicate successful regression, while residual correlations closer to one indicate failure of regression.

The self-regression tests demonstrated in the simulations could be applied to regression vectors used in in-vivo analysis. Criteria for the maximum number of spectral bands may be derived for any regression vector through self-regression tests. The process involves segmenting the whole-band regression vector into a varying number of spectral bands, performing regression of the whole-band regression vector using each set of the spectrally segmented regression vectors, correlating the resulting signals with the whole-band regression vector itself, and developing a correlation threshold for the residual correlation as a function of number of spectral bands. The maximum number of spectral bands could then be determined based on the tolerated residual correlation in the signal, i.e., on the maximum correlation below a preselected correlation threshold.

### Subjects

Resting-state scans were acquired in five male right-handed healthy controls aged 22 to 53 years, a male patient with a low grade glioma in insular, frontal and temporal cortex aged 46 years and a male patient with a glioblastoma in medial inferior temporal gyrus and temporo-occipital gyrus aged 54 years. Subjects were instructed to clear their mind and try not to think anything in particular, relax and fixate on a cross-hair presented on a computer screen during eyes open resting scans. One of the healthy controls was scanned during hypocapnic state induced by capnometry controlled hyperventilation (pETCO2: 19-25 mm Hg) to assess the robustness of the present method to increased and more variable levels of respiratory and cardiac signal pulsation.

### Data Acquisition

Data were collected at the Mind Research Network on a 3T Siemens TIM Trio scanner (Siemens Healthineers, Malvern, PA) equipped with MAGNETOM Avanto gradient system and 32-channel head array coil. High-resolution T₂-weighted Turbo Spin Echo scans were acquired for anatomical reference. FMRI data were acquired in AC/PC orientation using (a) multi-slab echo-volumar imaging (MS-EVI) (TR/TE: 246/25 ms, flip angle: 20°, no. of scans: 1000, 1100 or 1500, no. slabs: 4, spatial matrix per slab: 64×64×8, voxel size: 4×4×4 mm³, scan times: 4:10, 4:35 or 6:13 min; and (b) multi-band echo-planar imaging (MB-EPI) (https://www.cmrr.umn.edu/multiband/): TR/TE: 205/30 ms, flip angle: 15°, no. of scans: 3000, number of slices: 24, in-plane spatial matrix: 64×64, voxel size: 4×4×4 mm³, inter-slice gap: 0 mm, MB factor: 8, and scan time: 10:21 min. Real-time image transfer, motion correction, spatial normalization seed selection were performed. WM and CSF seeds for regression were manually delineated. The following atlas-based networks were mapped in subject-space: sensorimotor (SMN)-BA1-3, default mode (DMN)-BA7&31, visual (VSN)-BA17, and auditory (AUN)-BA41,42.

### Seed-Based Resting-State Connectivity Analysis in Turbofilt

Preprocessed in vivo data were analyzed in TurboFilt without the use of spatial smoothing. Respiration and cardiac pulsation frequency ranges for the in vivo data were specified based on concurrently acquired respiratory and cardiac waveforms collected during the scans. White matter masks were manually generated. The whole brain mask was generated based on a 30^{th} percentile intensity threshold applied to the time averaged brain image. Spectrally and temporally segmented regression of motion parameters and physiological noise was performed as described above. A conventional seed-based correlation analysis was performed and FIR filters were applied to assess connectivity in different frequency bands.

### Seed-Based Resting-State Connectivity Analysis in TurboFIRE

Spectrally and temporally segmented regression of motion parameters and physiological noise was performed in TurboFilt (see above). Preprocessed data were further analyzed in TurboFIRE using isotropic 5 mm Gaussian spatial smoothing, 8 s moving average time domain low pass filter with a 100% Hamming window width, and sliding window correlation using an 8 s window with moving averaging of up sliding-window correlation maps. Thresholded correlation maps (r=0.03-0.7) were obtained. A correlation threshold of 0.5 corresponds to a threshold of p<0.0001 (corrected for Family-wise error rates using an auto-regressive AR(3) model).

### Comparison With HRAN

The embodiments of the present invention were compared to that of HRAN using demonstration data provided with HRAN [https://github.com/LewisNeuro/HRAN] and motion-corrected data from in-vivo scans performed in the present work. In HRAN, the default parameters provided with the HRAN demo script were used: neural frequency=0.13 Hz representing the frequency of the visual task, a window length of 30 seconds with 75% overlap, cardiac range from 0.83 Hz to 1.3 Hz, respiration frequency range from 0.1 Hz to 0.3 Hz. The present method used 5 non-overlapping temporal segments (˜31 seconds each). Physiological noise (respiration and cardiac) was labeled automatically using the respiratory belt and cardiac rate data provided. The labels were created based on the minimum and maximum measured rates in each temporal segment. Regression vectors for respiration and cardiac pulsation were generated the same way described in the methods section through localizing labeled features and constructing an average signal. Spectrograms of the signals from the visual network were generated using the Chronux toolbox [http://chronux.org/]. Windows of 30 second length with 50% overlap were used in the denoising of in vivo data in HRAN.

### RESULTS

### Simulations

Connectivity in the 2-slice Rician brain model at low (<0.3 Hz) and high (>0.3 Hz) frequencies for 4 different seed regions is shown in FIG. 1. The posterior brain region seeds exhibit low frequency connectivity while the anterior seeds exhibit high frequency connectivity as intended in the model (FIGS. 1b and 1c). Low frequency connectivity in seeds with high frequency baseline correlations and high frequency connectivity in seeds with low frequency baseline correlations is limited to the seeds themselves, as expected. Whole frequency range noise (up to 2.43 Hz) based on motion parameters from an actual scan that was injected to the right brain slice and low-passed (<0.3 Hz) noise from the same motion parameters that was injected to the left slice significantly increased correlations. After noise injection all seed regions exhibit low frequency connectivity due to the shared noise <0.3 Hz. At high frequencies >0.3 Hz, the anterior left seed correlates with the anterior right seed as in the original noise-free model. The anterior right seed, however, correlates with both the anterior left seed and the posterior right seed where noise >0.3 Hz was also injected. The posterior right seed correlates only with the anterior right seed at frequencies above >0.3 Hz in the model with noise injection. The brain model with noise injection is used an input to the denoising algorithm to assess the ability of the algorithm to recover the original noise-free brain model at high and low frequencies.

The conventional approach which uses whole-band regression vectors (1 spectral band) is compared to the present approach which uses multiple spectral segments in FIG. 2. The conventional approach successfully removes the noise and recovers the original correlations when the regression vector matches the injected noise as in the case for seeds (ii) and (iv) in the right slice (Figures 1a and 1b). It, however, fails to recover the original signals when a whole-band regression vector is used to remove injected noise that had been low-passed as is the case for seeds (i) and (iii) in the left slice indicating its susceptibility to failure when uncertainties are present in part of the spectral domain.

The embodiments of the present invention eliminate the injection of artifactual correlations to high frequencies (>0.3 Hz) and recovers the original low frequency connectivity (<0.3 Hz) when a sufficient number of spectral bands are used. In the example in FIG. 2, nine uniform spectral bands are found to be sufficient for the recovery of the original correlations. When nine spectral bands are used, the bandwidth of each regression vector is 0.27 Hz. This allows the components of the signal >0.3 Hz to be regressed almost independently of the component of the signal <0.3 Hz. When an insufficient number of spectral segments are used, as in the case of 3 segments, artifactual connectivity can still be observed despite the improvement in denoising compared to using 1 spectral band. For the simulations in FIG. 2, ideal regression can be achieved by using 2 non-uniform spectral segments such that the first segment is <0.3 Hz and the second segment is >0.3 Hz. However, this requires that the spectral band of interest for connectivity analysis to be defined prior to denoising, which limits the usefulness of the post-processed data. Additionally, the performance of the denoising process will be undermined if uncertainties are present in a spectral segment of the regression vector within the spectral band of interest. Use of a sufficiently large number of uniform spectral bands mitigates these problems. It is, therefore, important to understand whether there is an upper limit on the number of uniform spectral segments that could be used.

Self-regression tests were conducted using different numbers of uniform spectral segments in order to explore the limitations on the number of spectral segments. The non-causal filter employed in the segmentation process effectively has no transition band which allows for larger numbers of spectral segments to be used. The regression vectors in self-regression tests were based on spectral segmentation of the original signal. FIG. 3 shows that the correlation between the original signal and the signal obtained from self-regression increases with increased number of spectral segments proportional to the natural logarithm of the number of spectral segments. A criterion could, therefore, be derived for the maximum number of spectral segments as:

$${N}_{bands} = floor ( \frac{1}{b} {e}^{\frac{T}{a}} ) \approx floor ( \sqrt{3} {e}^{30 T} ) \qquad ( 5 )$$

where T is the maximum tolerated residual correlations, which may be decided based on the correlation threshold used in the correlation maps. Residual correlations were below 0.1 when 32 segments of 0.039 Hz bandwidth were used. When 128 segments of 0.00976 Hz were used, residual correlations approached 0.15.

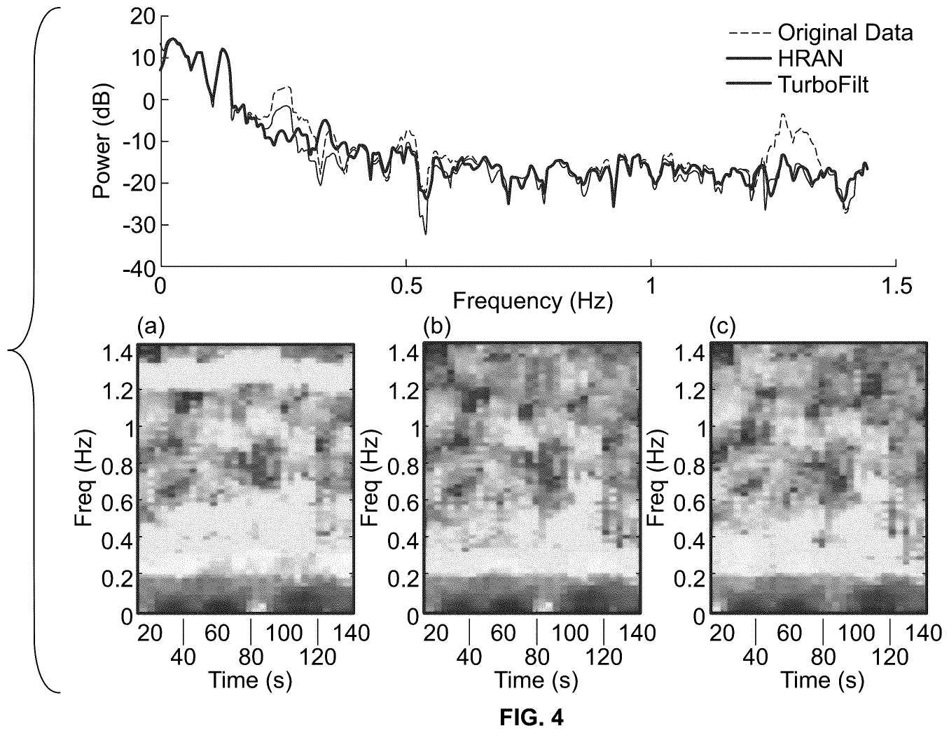

The comparison of spectrally and temporally segmented regression with HRAN using demonstration data provided with HRAN shows that both approaches have similar performance for removing respiratory and cardiac pulsation that are within narrow frequency ranges (FIG. 4). The respiratory belt data show that the respiration rate varied between 0.18 and 0.37 Hz with the 25^{th} and 75^{th} percentile being 0.24 and 0.275 Hz, respectively. The cardiac rate varied between 0.89 to 1.65 Hz but was mostly between 1.22 Hz (5^{th} percentile) and 1.38 Hz (95^{th} percentile) with the 25^{th} and 75^{th} percentiles being 1.26 Hz and 1.30 Hz suggesting that the cardiac rate was stable between 1.26 and 1.3 Hz for half the time. The spectra for a visual cortex seed obtained using the mask provided with the demo data show that both HRAN and spectrally and temporally segmented regression effectively take out cardiac pulsation. Differences are observed in respiration where greater suppression of the signals in the 0.2-0.3 Hz range is apparent in the case of spectrally and temporally segmented regression implemented in TurboFilt. The spectra also show that HRAN and TurboFilt do not appreciably alter data outside the specified physiological noise search ranges for HRAN or labels in different segments in case of TurboFilt.

### TurboFilt Analysis of In-Vivo Experiments

A significant reduction in artifactual correlations due to motion, respiration and cardiac pulsation using spectrally and temporally segmented regression was obtained in all subjects studied. A comparison of spectrally and temporally segmented regression with HRAN using resting-state fMRI data from a subject during hypocapnic state with increased head movement and physiological noise induced by hyperventilation demonstrates the tolerance of the present approach to signal fluctuations that cover a wide spectral range. Respiration in this data is shifted to relatively higher frequencies up to 0.75 Hz due to hyperventilation.

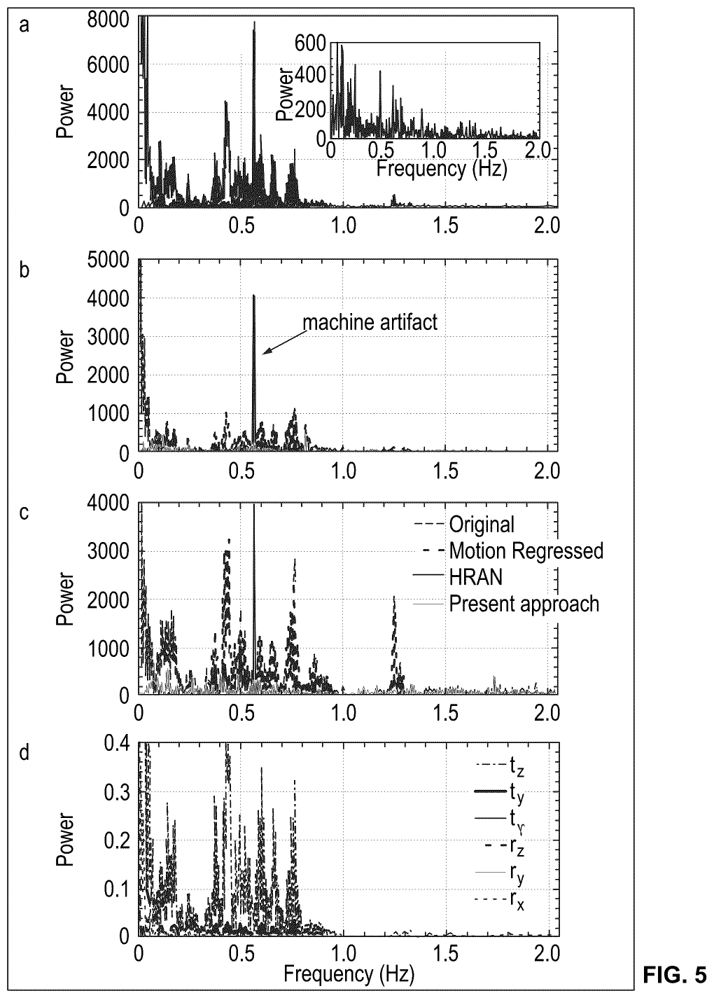

A machine artifact was present at 0.52 Hz. Through inspection of the motion spectra in (FIG. 5.d) and comparison with the original pre-motion regression spectra from ROIs in the visual network (FIG. 5a) and CSF (FIG. 5c), it appears that much of the low frequency part of the CSF and VSN signals are contaminated by translation in the z direction. The motion regression step substantially denoises signal components <0.2 Hz in the visual network and CSF. However, motion parameter regression does not take out respiration or cardiac signals in this case which justifies the physiological noise correction step. Both HRAN and spectrally and temporally segmented regression successfully take out cardiac pulsation in the CSF, but present approach does a slightly better job of suppressing respiration (FIG. 5c). Good agreement is observed between HRAN and the present approach in the visual network and default mode network ROIs with the exception that HRAN fails to remove the machine artifact that is present at 0.52 Hz that happens to be present in the middle of the respiration frequencies. The fact that the present approach removed the machine artifact suggests that it was coincidentally present in much of the regions in the brain from which the regression vectors were generated for regressing respiration, whereas it was less represented in the white matter region that HRAN uses to build models for respiration. In the denoised signals, lower frequencies tend to have higher magnitude of power spectral density than higher frequency ones (see the embedded figure in FIG. 5a) as expected from hemodynamic response models of the BOLD effect and detection of task-based fMRI signal changes at frequencies above 0.3 Hz.

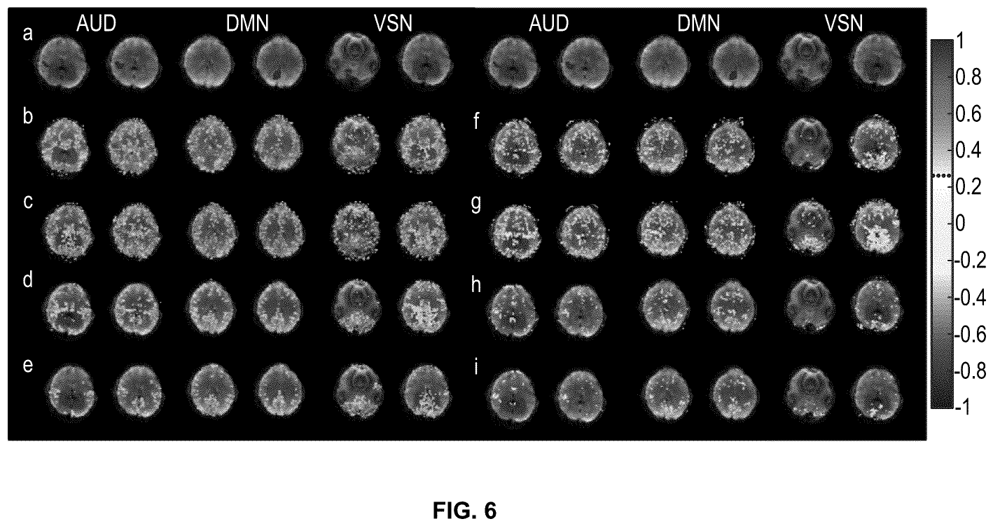

The detection of resting-state networks at different stages of spectrally and temporally segmented regression and in different frequency bands is shown in FIG. 6. Unilateral seed regions are used for auditory, default mode, and visual networks (FIG. 6a). For spectral and temporal segmentation case, a total of 12 uniform spectral segments were used with 0.169 Hz width each. The time domain was divided into 8 segments (33.7 seconds each). Full width correlations are calculated for data in the entire frequency spectrum (0-2.03 Hz), which are dominated by low frequency connectivity (FIGS. 6b-6e). Before motion regression, substantial noise is present in the correlations as manifested in significant correlations near the edges and far from the expected hubs of the resting-state networks (FIG. 6b). Full-width, whole-band regression of motion parameters without high pass filtering fails to take out low-frequency confounds (FIG. 6c). The addition of a FIR high-pass filter to full-width whole-band regression with a pass-band of 0.005 Hz and a stop-band of 0.03 Hz helps remove the low-frequency confounds that result mainly from motion (FIG. 6d). The connectivity maps in FIG. 6.d are considerably cleaner than those in FIG. 6c and have less edge artifacts. Spectrally and temporally segmented regression of motion parameters without FIR high pass filtering (FIG. 6e) produces substantially cleaner connectivity maps, even compared to full-width whole-band motion regression with FIR high-pass filter (FIG. 6d). Residual noise is present near the edges but is considerably reduced compared to the other cases. Bilateral connectivity is observed in all networks in FIGS. 6d and e although unilateral seeds are used. High frequency connectivity >0.3 Hz (FIGS. 6f-fi) appears spatially sparse compared to low-frequency dominated whole-band connectivity (FIGS. 6b-6e) even prior to motion regression (FIG. 6f), as expected. An important observation is the introduction of artifactual connectivity in the visual network at frequencies above 0.3 Hz in the case with full-width, whole-band motion regression (FIG. 6g). The connectivity observed is not present in the case without motion regression (FIG. 6f) and is also absent when spectrally segmented regression is used. Artifactual connectivity in whole-band regression is injected as a result of a partial match of the regression vector in part of the spectral domain with the signals in the visual network. This artifactual connectivity is not present when spectrally segmented regression is used (FIG. 6h) as there are no shared weights between different spectral segments in regression. Weaker connectivity is observed compared to whole-band regression in the auditory and visual networks when spectrally segmented regression is used. No substantial difference is observed between spectrally segmented regression (FIG. 6h) and spectrally & temporally segmented regression (FIG. 6i) for connectivity >0.3 Hz. Maps produced from the latter are slightly cleaner.

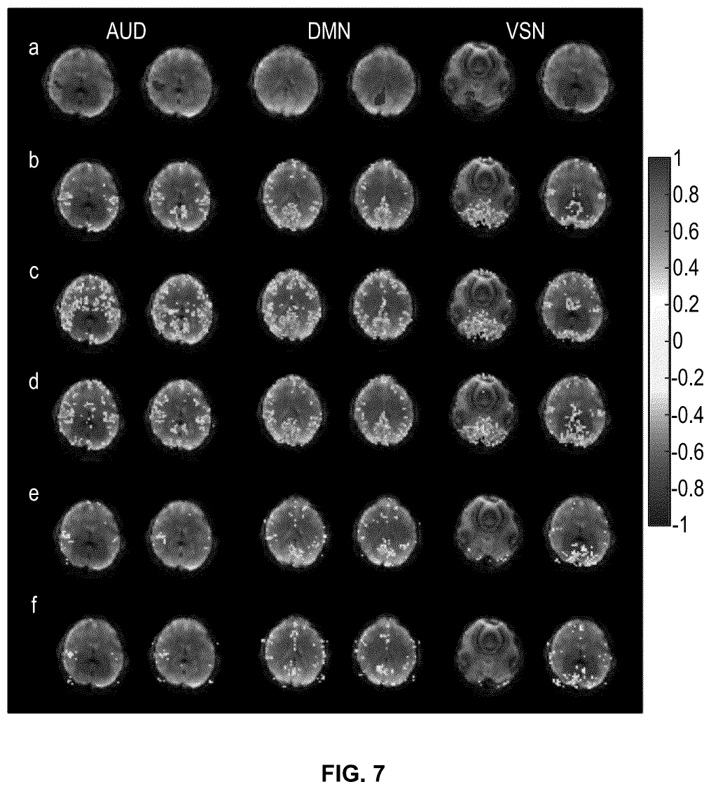

FIG. 7 shows a comparison of physiological noise regression using spectrally and temporally segmented regression implemented in TurboFilt with HRAN for both low frequency dominated connectivity (FIGS. 7.b-d) and high frequency connectivity >0.3 Hz (FIGS. 7e and 7f). The input to the physiological noise regression step in TurboFilt is motion regressed data obtained through spectrally and temporally segmented regression of the 6 translation and rotation parameters. Strong bilateral resting state networks are observed for auditory, default mode, and visual networks with little edge noise in the case of TurboFilt (FIG. 7b). Two cases were considered for HRAN in order to: (a) compare HRAN and TurboFilt protocols, (b) compare physiological noise regression capability only. In the first case for HRAN (FIG. 7c), a high pass filter >0.03 Hz is used as a pre-step to remove low-frequency motion confounds. No regression of motion parameters is employed. The maps in FIG. 7c are noisier compared to those in FIG. 7b with substantially more correlations near the edges and outside the networks. This highlights the importance of the spectrally and temporally segmented motion regression step in TurboFilt. Comparing the results in FIGS. 7d and 7b, physiological noise correction in HRAN produces comparable results with that in TurboFilt although the maps in the auditory network in the case of HRAN are slightly more contaminated with noise. Similar results are observed in DMN and VSN. High frequency connectivity >0.3 Hz in the auditory network without spatial smoothing is not detectable using TurboFilt (FIG. 7e) and HRAN (FIG. 7f). Significant bilateral high frequency connectivity in multiple slices is observed in the default mode network, even without spatial smoothing. Localized bilateral connectivity is observed in one slice in the visual network using a unilateral seed region.

### TurboFire Analysis of In-Vivo Experiments

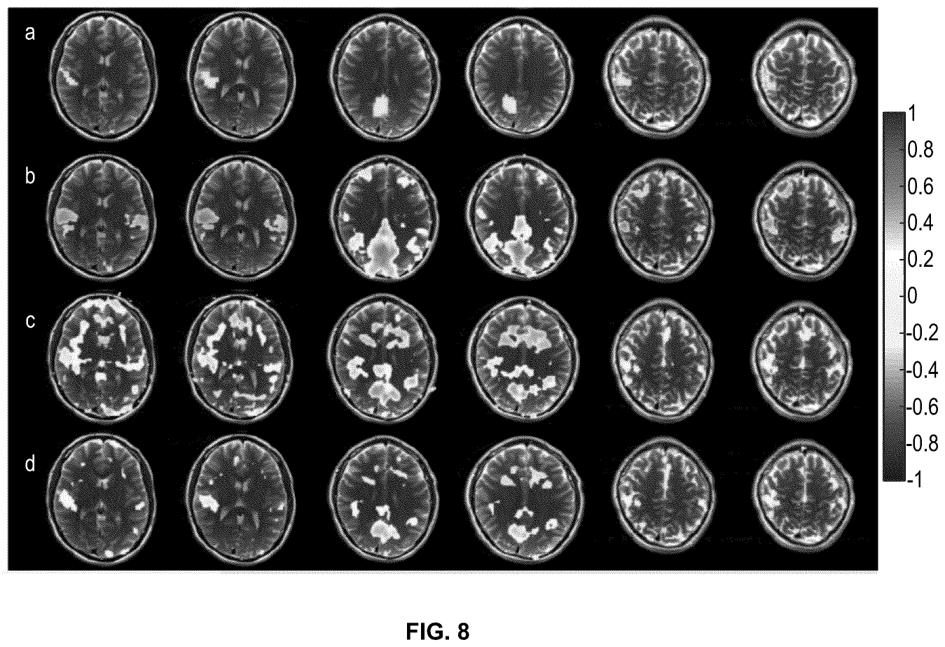

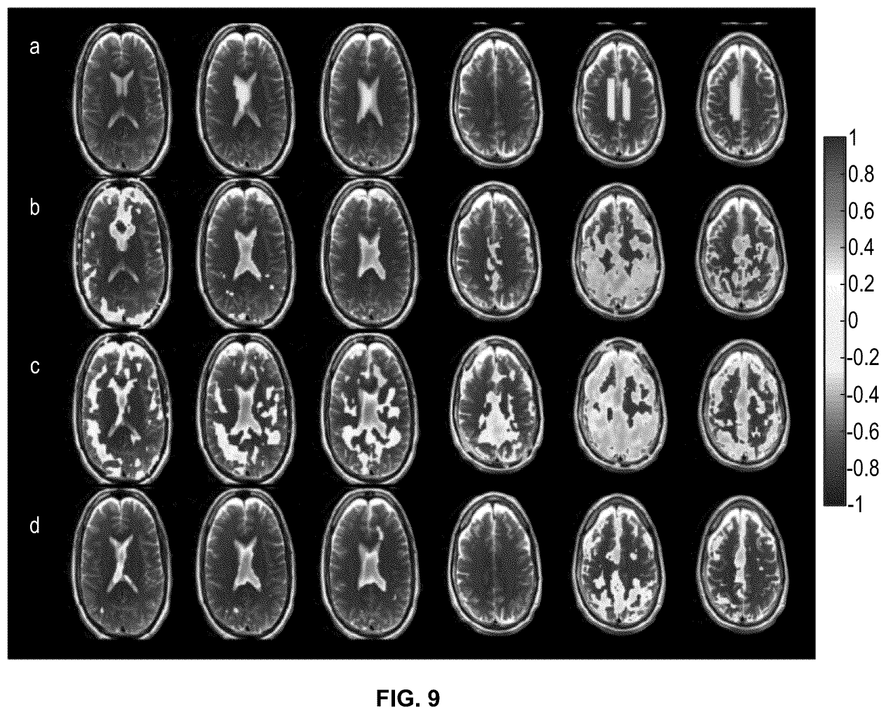

Combination of spectrally and temporally segmented regression with averaged sliding window correlation further enhances confound suppression at frequencies above 0.3 Hz. Spectrally and temporally segmented regression in a healthy control shows bilateral connectivity in the auditory, default mode and sensorimotor networks at frequencies above 0.3 Hz (FIG. 8d) with reduced artifactual connectivity compared with HRAN (FIG. 8c). Default mode connectivity in inferior parietal lobule (IPL) region is visible at high frequencies. High frequency connectivity is co-localized with low frequency connectivity (FIG. 8b). Using data acquired in a healthy control during hyperventilation shows that spectrally and temporally segmented regression of movement parameters and physiological signal pulsation is effective at reducing artifactual connectivity at high frequencies (>0.3 Hz) outside of major resting state networks (FIG. 9d) compared with HRAN (FIG. 9c) and low frequency connectivity (FIG. 9b). These results are consistent with the reduction in motion and physiological noise in the spectra shown in FIG. 5 that were obtained in the same subject.

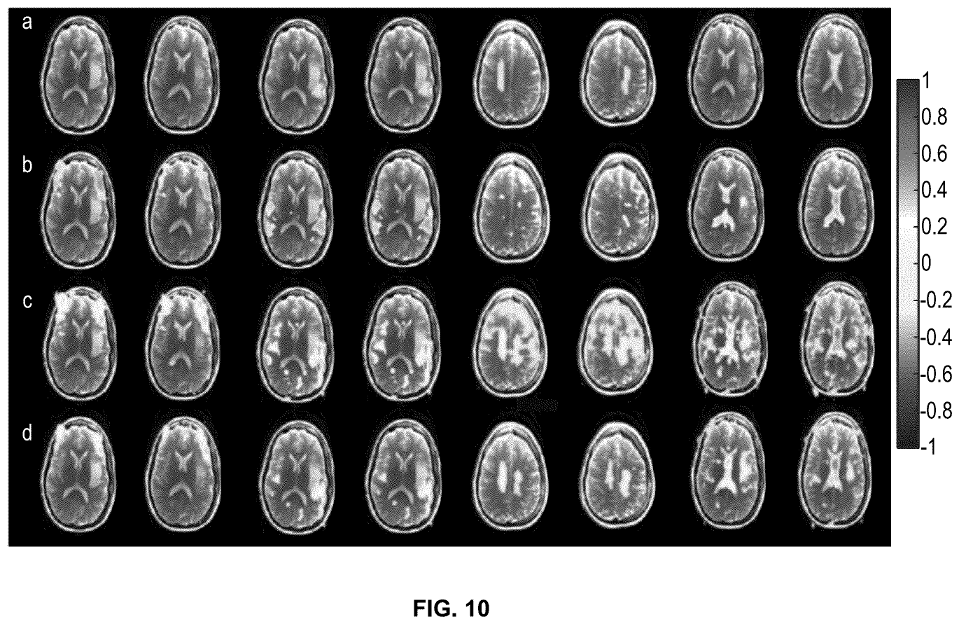

Connectivity at high frequencies in the language and auditory networks was also detected in the two patients with brain tumors. Spatial displacements due to the tumor was seen at low and high frequencies when analyzed with unilateral seeds. FIG. 10 shows bilateral language connectivity and a posterior displacement of the auditory network on the tumor side in the patient with the low grade glioma in insular, frontal and temporal cortex at high frequencies, consistent with the localization seen at low frequencies. The WM seed shows increased reduction of physiological noise in WM and GM areas using spectrally and temporally segmented regression compared to HRAN (FIGS. 10c and 10d). The reduction of physiological noise correlated with CSF pulsation using HRAN and spectrally and temporally segmented regression was comparable and showed only minor artifactual correlations outside of CSF spaces (FIGS. 10c and 10d).

### Computational Performance

Motion regression of an MS-EVI data set with 1100 time points (scan time: 4:35 min) in TurboFilt using 12 spectral bands applied to 3 translation and 3 rotation parameters and 8 temporal segments (33.7 sec each) took 48.6 seconds per segment on a 2.30 GHz, 2×18 core Linux workstation with 64 GB RAM running on one CPU thread. Total processing time for motion regression was 389 seconds. Physiological noise regression for this data set took 69 seconds per segment, resulting in a total processing time for physiological noise regression with phase shifting of 552 seconds. The total number of motion regression vectors therefore was 73 vectors per temporal segment (6*12+1(constant vector)=73). Therefore, the processing time per temporal segment for both motion and physiological noise regression was 117.6 seconds, resulting in a total processing time of 940.8 seconds.

### DISCUSSION

Spectral and temporal segmentation of motion parameters and physiological noise signals represents a powerful and novel regression approach for minimizing confounding non-neuronal signal fluctuations in resting-state fMRI data. As results demonstrate, the approach is particularly powerful for removing head movement and respiration related signal changes that cover extended frequency ranges that cannot approximated by single frequency regression vectors. Data also show that this approach can be combined with averaged sliding window correlation analysis to further enhance confound suppression. This methodology preserves neuronal signal fluctuations of interest that are underlying the confounding signals and thus reduces data loss compared to FIR filtering, which takes out all signal in brain regions affected by movement related signal changes and physiological noise. Based on the simulations, the original signals and correlations appear recovered and not lost. In the real world, the effectiveness of the regression process will depend on the extent to which the regression vectors represent the local noise.

Rather than deciding a band of interest prior to processing which limits the usefulness of the post-processed data, systematic segmentation into many segments can achieve similar independence while allowing the data to be used to explore high frequency connectivity in arbitrary bands after processing. This is also advantageous because if the band of interest is too broad, there may be uncertainties in the regression vectors in part of the band. Segmentation into many spectral segments was shown here to effectively solve this problem with little downside as demonstrated in the self-regression tests. Co-linearity of motion parameters and respiratory signal pulsation may be present in the data, resulting in double regression. Generally, double regression should have no effect on the suppression of confounding signals as a zero weight would be calculated for the second regression vector.

### Simulations

The artifactual high frequency correlations in FIG. 2 are seen exclusively in regions where only the low frequency component (<0.3 Hz) of the noise was injected (FIG. 1). No artifactual correlations are observed in the background or in other regions where low-passed motion parameters were not injected as the regression weights are nearly zero. This indicates that the artifactual correlations follow from the biasing of the weighting coefficients during regression by the matching low frequency part of the signals and the regression vectors, while a mismatch is present in the high frequency part. As only one spectral band is used in conventional regression, it does not assign different weights to different spectral bands resulting in artifactual connectivity in spectral bands where connectivity is neither present in the original model nor the noised model.

The simulation of whole bandwidth regression in FIG. 2 demonstrates the inability of the conventional regression approach to denoise the signals when uncertainties are present in the regression vector in a portion of the spectral domain. Notably, when regression fails, false-positive high frequency connectivity is injected, and low frequency correlations are not successfully recovered as observed in the connectivity maps for seeds (i) and (iii) in FIG. 2. Our observations are consistent with the injection of artifactual connectivity in resting-state networks in whole bandwidth regression demonstrated by Chen et al23. However, it was found in the present study that the artifactual networks observed in high frequencies do not necessarily represent low frequency connectivity, but rather regions where low frequency nuisance present in the signals (e.g., due to motion) matched better with the regression vectors than the high frequency components of the noise.

The results in FIG. 3 suggest that using an excessively large number of spectral segments can weakly compromise the effectiveness of the regression process especially when the regression vectors can be measured with high accuracy. On the contrary, the results in FIG. 2 show that increasing the number of segments is beneficial when uncertainties are present in part of the spectral domain of the regression vectors. Therefore, the recommendation is to use the maximum number of uniform spectral bands constrained by the tolerated level of residual correlations due to spectral segmentation.

When combined with temporal segmentation, the number of spectral bands may be additionally limited by the length of the temporal window in order for the system of equations not to be overdetermined. For this reason, phase shifting of regression vectors in physiological noise regression that is based on minimizing the power spectral integral in the frequency range of the labeled nuisance feature in the corrected signal across the entire slice is preferred to using a fixed array of phase shifts. Analyses performed during the development of the method showed that nearly similar results could be obtained using a fixed array of phase shifts with no iterative minimization but at the expense of substantially increasing the number of regression vectors.

For full-width whole-band regression to work, confounds in the signals should precisely match the regression vector23. The performance of full-width whole-band regression is significantly affected by uncertainties and/or delays in the measured motion parameters in any part of the frequency spectrum as demonstrated in FIG. 6 and also in the simulations in FIG. 1. An example of such uncertainties is intra-scan motion that manifests as non-rigid head motion, which can result in mismatches in part of the spectral domain between measured motion parameters and noise in the signals. Differences between different regression techniques may be exploited to identify regions with similar tissue properties (since there would be artifactual connectivity in those areas).

### Detection of Signal Correlations at Frequencies Above 0.3 Hz

The methodology was developed to improve detection of signal changes above the traditional upper frequency limit of resting-state fMRI, which we have reported recently40. Given the considerable overlap between respiratory frequencies and the expected neuronally driven high-frequency signal changes it is not desirable to use FIR filter that would remove considerable portions of high-frequency connectivity. Furthermore, our study design in an ongoing related study is aimed at detecting changes in high-frequency connectivity as a function of global blood flow changes induced by hypo- and hypercapnia^{41,42}, which increases respiratory signal pulsation. As our data in a subject undergoing hyperventilation show, although respiratory signal changes increase and spread over a larger spectral range than in resting-state fMRI data acquired during normocapnic conditions, spectral and temporal segmentation of motion parameters and physiological noise signals in conjunction with averaged sliding window correlation analysis strongly reduces confounding non-neuronal signal fluctuations in high-frequency resting-state fMRI data. The results shown in FIGS. 8-9 provide evidence for resting-state networks at frequencies above 0.3 Hz in healthy controls. Consistent with the results shown in a patient with a brain tumor in⁴⁰ our present data confirm that spatial displacement of resting-state connectivity due to a tumor is consistent at low and high frequencies, which supports the physiological origin of high frequency connectivity.

### The TurboFilt Software Tool

TurboFilt is a multi-purpose, MATLAB-based framework developed in-house for the post-processing of fMRI data. The framework primarily serves as a graphical user interface-enabled test-bed for new methods for denoising and analyzing reconstructed fMRI signals. The code features functions for robust seed selection, spatial mask generation and application, conventional FIR filtering, time-course extraction and frequency analysis, spectral feature mapping, seed-based connectivity analysis with on-the-fly frequency range selection, principal component analysis, Rician noise simulation, motion regression, and physiological noise regression. TurboFilt enables multiple datasets to be processed within the same session. The present method has been implemented in the framework with a tutorial available in the TurboFilt manual.

While the foregoing written description enables one of ordinary skill to make and use what is considered presently to be the best mode thereof, those of ordinary skill will understand and appreciate the existence of variations, combinations, and equivalents of the specific embodiment, method, and examples herein. The disclosure should therefore not be limited by the above-described embodiments, methods, and examples, but by all embodiments and methods within the scope and spirit of the disclosure.

What is claimed is:

1. A method of performing functional magnetic resonance imaging, comprising the steps of:
a. Measuring and reconstructing a time series of high-speed functional MRI scan data sets in human or animal brain using an acquisition rate that enables spectral separation of physiological noise signals, including, but not limited to respiratory and cardiac pulsatility, and hardware related signal fluctuations;
b. Measuring a plurality of rigid body movement parameters for translations and rotations in different directions in said data sets relative to one of said data sets;
c. Spatially segmenting said data sets into multiple brain regions, including, but not limited to gray matter, white matter and cerebrospinal fluid containing regions;
d. Measuring said physiological noise signals and said hardware related signal fluctuations in said regions;
e. Spectrally and temporally segmenting said motion parameters, said physiological noise signals and said hardware related signal fluctuations, resulting in multiple regression vectors for each spectral segment and each of said regions;
f. Temporally segmenting said functional MRI scan data sets into said temporal segments; and
g. Performing linear regression within each temporal segment and spatial region of said data sets using said regression vectors.

2. The method of claim 1 further comprising the step of minimizing the loss of intrinsic resting-state signal fluctuations and mitigating the introduction of false positive signal fluctuations by the segmentation of said regression vectors in the spectral and temporal domain.

3. The method of claim 1 further comprising the step of segmenting said scan data in time into n segments within which frequency and amplitude fluctuations of motion parameters and physiological noise are reduced compared to the entire scan.

4. The method of claim 1 further comprising the step employing a temporally segmented regression of said scan data using spectral segmentation of said motion parameters, said physiological noise signals and said hardware related signal fluctuations into k spectral segments with no overlap; said scan data is segmented in time into n segments with no overlap and the length of each segment is chosen such that it is long enough to resolve features in the spectral domain and short enough for respiration rate, cardiac pulsation and hardware related signal fluctuations, to be sufficiently stable.

5. The method of claim 1 further comprising the step of employing a sliding window for temporal segmentation.

6. The method of claim 1 wherein regression vectors are constructed from said motion parameters and their higher order derivatives; each motion parameter is filtered into k number of segments in the spectral domain to obtain an independent regression vector for each spectral band.

7. The method of claim 6 wherein a non-causal filter is used to filter the motion parameters by applying FFT to the motion parameter time course then using inverse FFT to obtain the segment of interest.

8. The method of claim 7 wherein a temporally segmented regression of said filtered motion parameters is applied to obtain motion-corrected data.

9. The method of claim 1 wherein a spatial mask of the labeled feature is generated based on an anatomical brain atlas and used to obtain a spatially averaged signal to construct a representative regression vector.

10. The method of claim 1 wherein a mask is generated based on a power-spectral integral threshold relative to a labeled non-physiological noise frequency range.

11. The method of claim 10 wherein the average signal within the mask is pass-band filtered in the frequency range of the labeled feature to construct a regression vector for the feature within the segment.

12. The method of claim 1 wherein constructed regression vectors are phase shifted individually for each slice of said functional MRI scan data sets to minimize the power spectral integral in the frequency range of the feature in the corrected signal across the entire slice.

13. The method of claim 1 wherein the maximum number of spectral bands is determined using self-regression testing where regression vectors are segmented into a varying number of spectral bands and regressed using each set of spectrally segmented regression vectors with the maximum number of spectral bands decided based on the tolerated residual correlation between the original regression vector and resulting regressed signal.
